(19) United States
(12) Patent Application Publication
(10) Pub. No.: US 2004/0204733 A1
(43) Pub. Date: Oct. 14, 2004

(54) Surgical needle assembly comprising a stopper

(76) Inventor: Patrick J. Ferguson, Portland, OR (US)

Correspondence Address: GLENN C. BROWN, PC, 777 NW WALL STREET, SUITE 308, BEND, OR, 97701, US

(21) Appl. No.: 10/835,871

(22) Filed: Apr. 29, 2004

Related U.S. Application Data
(63) Continuation-in-part of application No. 10/407,803, filed on Apr. 4, 2003.
(60) Provisional application No. 60/370,546, filed on Apr. 4, 2002.

(51) Int. Cl. A61M 5/178; A61B 17/34

(52) U.S. Cl. 606/185

(57) ABSTRACT

A stopper for holding one part of a surgical needle assembly approximately fixed relative to another part. The stopper includes a planar rectangular body having two opposing faces and a protrusion that extends away from the body. The stopper may also include a groove that extends from the base of the protrusion to the top edge of the body. Each face typically includes a central depression configured for gripping by a finger or thumb. A needle assembly, for use with the stopper of the present invention, typically includes a hollow needle with a gripping member mounted on its proximal end, and a push rod configured to slidingly engage the hollow needle. The stopper is typically configured to fit snugly between the gripping member and the push rod to prevent movement of the push rod relative to the hollow needle during use.

### CROSS-REFERENCE TO RELATED APPLICATIONS

This application is a continuation-in-part of U.S. patent application Ser. No. 10/407,803, filed on Apr. 4, 2003, which is a continuation of U.S. provisional patent application No. 60/370,546, filed on Apr. 4, 2002. The priority of these prior applications is expressly claimed and their disclosures are hereby incorporated by reference in their entirety.

### BACKGROUND OF THE INVENTION

1. Technical Field

The present invention relates generally to surgical tools and, more specifically to a stopper useful for holding a stylet of a surgical needle assembly in a fixed position relative to a cannula.

2. Field of the Invention

Hollow needle/push rod assemblies can be used for a variety of medical applications and procedures. Typically the push rod, or stylet, fits within, and is free to move along the longitudinal axis of the hollow needle, or cannula. Such hollow needle/push rod assemblies often are used when depositing, sampling or removing material within living tissue, for example in brachytherapy procedures.

The detailed description of the invention, below, is based on an embodiment of the present invention that may be used in prostate brachytherapy, in which radioactive seeds are implanted in a human prostate while the needle and seeds are monitored by ultrasound sensors and imaging devices. U.S. Pat. Nos. 4,461,280, 4,700,692, and 4,815,449, the disclosures of which are incorporated herein by reference, disclose background information about the deposition of radioactive seeds within human tissue.

As discussed below, manipulation of loaded hollow needle/push rod assemblies can be difficult, requiring a user to perform several assembly-related tasks simultaneously. During assembly preparation, the distal end of the hollow needle is plugged using a needle plug material that is known in the art. The plug prevents the seeds and spacers from inadvertently ejecting from the distal end of the needle during loading. The radioactive seeds or seeds disposed between spacers are loaded into the cavity of the hollow needle through a gripping member located at the proximal end of the needle. In order to facilitate the seed loading, the inside surface of the gripping member has a frustroconical or funnel-like shape. After the seeds are loaded into the hollow needle, the push rod is inserted into the hollow needle behind the seeds so that the distal end of the push rod is adjacent to the last seed or spacer.

In operation, the push rod, or stylet, is used to push the seeds out of the cannula, or needle, into a desired position in the organ. Whether the needle is loaded or unloaded, the push rod is typically free to move within the needle assembly, as dictated by gravitational forces. Thus, to avoid unwanted seed ejection or movement of a loaded assembly, the push rod typically must be manually held in a fixed position (relative to the needle). A user is therefore burdened not only with performing sensitive injection manipulation during seed implantation, but also with holding the push rod and needle in a fixed orientation so that the seeds don't inadvertently fall out.

### SUMMARY OF THE INVENTION

A stopper is provided that is useful for holding one part of a surgical needle assembly approximately fixed relative to another part. The stopper typically includes a planar body having two opposing faces and a protrusion that extends away from the body. Each face typically includes a central depression configured for gripping by a finger or thumb. Alternatively, the planar body may contain a cylindrical opening, located at its center.

A needle assembly, for use with the stopper of the present invention, typically includes a hollow needle with a gripping member mounted on its proximal end, and a push rod configured to slidingly engage the hollow needle. The gripping member has a tapered inner surface.

The stopper is configured such that the protrusion is cylindrical in shape, narrowing slightly as it extends away from the body. The tapered shape of the protrusion fits snugly between the push rod and the tapered inner surface of the gripping member to prevent movement of the push rod relative to the hollow needle during use. Additionally, the stopper is configured such that it may be easily removed from the needle assembly when ready for use. The stopper can be removed without disturbing the position of the push rod.

In an alternate embodiment, the stopper also comprises a groove that extends from the base of the protrusion to the top of the body. The width of the groove is determined by the width of the push rod. The groove of the stopper is typically configured to almost completely surround the push rod so that the stopper, when engaged with the push rod, fits snugly within the gripping member to prevent movement of the push rod relative to the hollow needle during use. The stopper is removeably attached to the push rod by pressing the cylindrical push rod into the cylindrically shaped groove of the stopper. The stopper is formed of a flexible, rubber-like material that releaseably grips the push rod.

### BRIEF DESCRIPTION OF THE DRAWINGS

FIG. 1 is a view of a stopper, according to one embodiment of the present invention.

FIG. 2 is a surgical needle assembly.

FIG. 3 depicts the surgical needle assembly for use with the stopper shown in FIG. 1.

FIG. 4 is a view of a stopper, according to an alternate embodiment of the present invention.

FIG. 5 depicts the surgical needle assembly for use with the stopper shown in FIG. 4.

### DETAILED DESCRIPTION OF THE PREFERRED EMBODIMENT

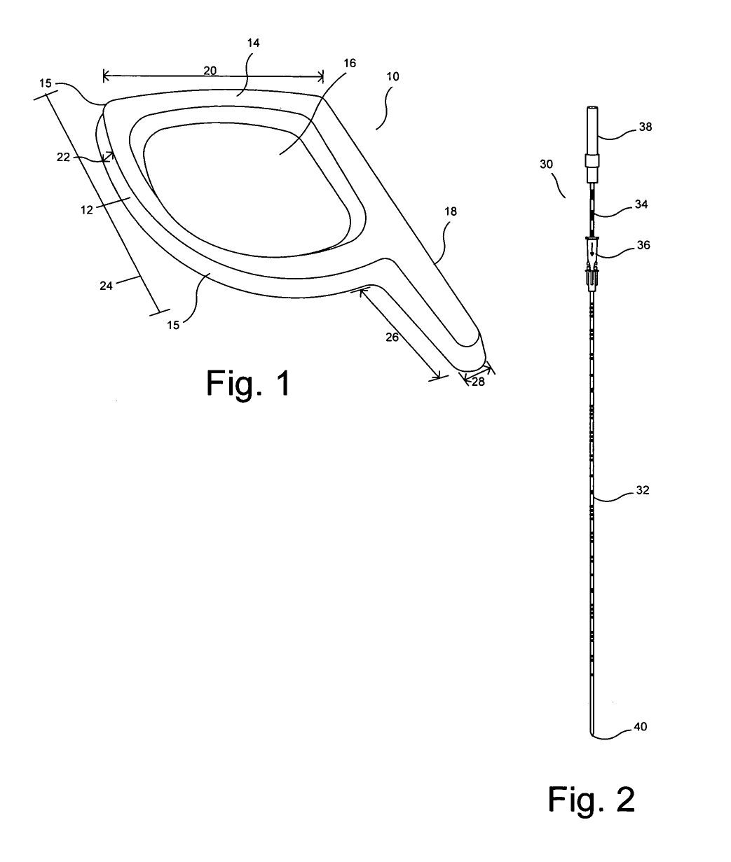

Referring initially to FIG. 1, a stopper for use with a surgical needle assembly, according to one embodiment of the present invention in generally shown at 10. Stopper 10 typically includes a substantially planar body 12 having two opposing faces, one face being shown generally at 14. Each face typically includes depression 16 configured to facilitate gripping of stopper 10 by a user. In an alternative form of the present invention, planar body 12 may contain a cylindrical opening, located at its center. Stopper 10 also typically includes protrusion 18 that is configured to hold one part of a surgical needle assembly approximately fixed relative to another part. Typically, the body 12 has rounded edges 15 and protrusion 18 is cylindrical in shape, narrowing slightly as it extends away from body 12.

Typically, planar body 12 is approximately 16 to 20 mm wide, 2 to 4 mm thick, and 12 to 16 mm long, as shown respectively at 20, 22, and 24. In a preferred embodiment, planar body 12 is 18 mm wide, 3 mm thick, and 15 mm long. Protrusion 18 typically extends from and is parallel to planar body 12. In a preferred embodiment of the present invention, protrusion 18 is flush with one side of body 12. Alternatively, protrusion 18 may be oriented in any other configuration, with respect to body 12 that allows stopper 10 to hold a push rod in a fixed position relative a hollow needle, as described above. Protrusion 18 is typically approximately 4 to 6 mm long and 1 to 2 mm wide as shown at 26 and 28, respectively. In a preferred embodiment protrusion 18 is 5 mm long and 1.3 mm wide.

Referring next to FIG. 2, a surgical needle assembly for use with a stopper 10 is shown generally at 30. Assembly 30 typically includes hollow needle 32 and push rod 34. Needle 32 is typically cylindrical in shape, its length being substantially greater than its diameter. Hollow gripping member 36 is typically mounted on the proximal end of needle 32. Gripping member 36 and hollow needle 32 are collectively referred to as a cannula. Member 36 is typically conical in shape, is composed of a rigid plastic material, and is configured such that it can be easily gripped by a finger and/or thumb. The inside surface of the gripping member 36 is tapered. Push rod 34 typically fits within the hollow cavity of needle 32 and is configured to slide freely within needle 32 to deposit material stored therein. Push rod 34 will be removed from the needle cavity when material, such as seeds and spacers are loaded into the cavity. Typically, push rod 34 does not engage gripping member 36. However, handling element 38, mounted on one end of push rod 34 is configured to engage the gripping member 36 as push rod 34 slides toward needle tip 40 of hollow needle 32. When gripping member 36 and handling element 38 are engaged, push rod 34 typically extends completely through the cavity of needle 32 to deposit loaded material. The material deposited is typically radioactive seeds or radioactive seeds disposed between spacers, useful for treating tumors.

In most orientations, gravitational forces cause push rod 34 to either fall out of hollow needle 32 or slide towards tip 40 until handling element 38 engages gripping member 36, as described above. Holding push rod 34 stationary at a fixed position, relative to needle 32, is typically accomplished by manually holding push rod 34, needle 32, or both. This creates an additional burden on a user who must manipulate the assembly for seed implantation while simultaneously maintaining push rod 34 in its fixed orientation.

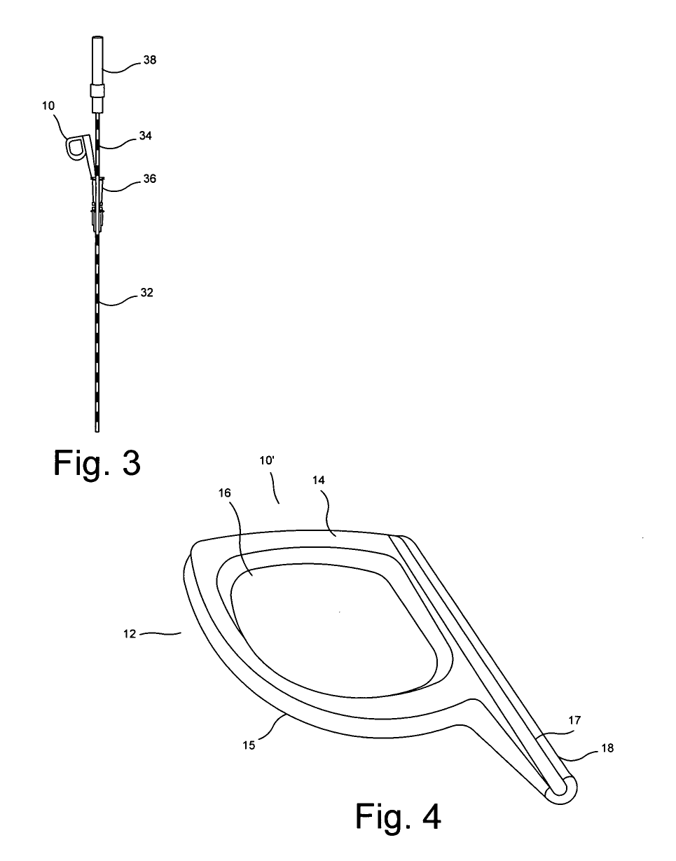

Referring next to FIG. 3, the assembly of FIG. 2 is shown including a stopper 10 according to the embodiment shown in FIG. 1. With the push rod 34 partially retracted to a desired position from the hollow needle 32, the stopper 10 is wedged into the gripping member 36 alongside the push rod 34. The protrusion 18 of the stopper 10 fits snugly between gripping member 36 and push rod 34, substantially limiting push rod 34's movement with respect to needle 32. Typically, stopper 10 is configured to hold push rod 34 in any fixed position, relative to needle 32, as long as the push rod is at least partially within the needle cavity. Maintaining push rod 34 in a fixed position reduces the tasks required for use of hollow needle/push rod assembly 30 and other similar types of assemblies. Thus, the user is free to concentrate on assembly manipulation, which improves the precision and consistency of seed implantation during brachytherapy.

In an alternate embodiment, the stopper 10 comprises a groove that is configured to releaseably grip and almost surround the push rod 34. Referring to FIG. 4, the grooved stopper 10′ is similar to the stopper 10 in FIG. 1 with the addition of the groove 17. The groove 17 is configured to hold push rod 34 in a fixed position relative to another part. The groove 17 extends from the base of the protrusion 18 to the top edge of the body 12. To accommodate the push rod 34, groove 17 is cylindrical in shape and has a diameter that is about equal to the outside diameter of the push rod 34.

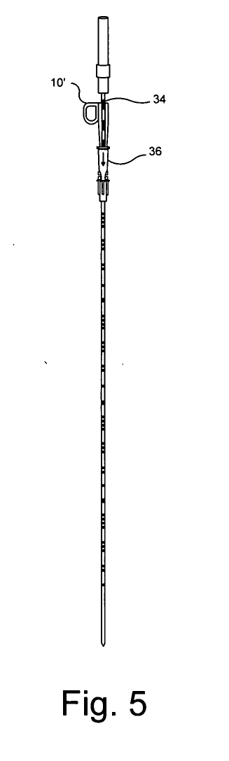

Referring next to FIG. 5, the assembly of FIG. 2 is shown including a stopper 10′ according to the embodiment shown in FIG. 4. The groove 17 of the stopper 10′ substantially surrounds the push rod 34. The protrusion 18 of the stopper 10′ fits snugly between gripping member 36 and push rod 34, substantially limiting push rod 34's movement with respect to needle 32. Typically, stopper 10′ is configured to hold push rod 34 in any fixed position relative to needle 32 as long as the push rod is at least partially within the needle cavity. Maintaining push rod 34 in a fixed position reduces the tasks required for use of hollow needle/push rod assembly 30 and other similar types of assemblies. Thus, the user is free to concentrate on assembly manipulation, which improves the precision and consistency of seed implantation during brachytherapy.

In another aspect of the invention, when the surgeon is ready to operate push rod 34, e.g. to eject radioactive seeds in the example of brachytherapy, the stopper 10 is gripped by the planar body 12 and pulled free and discarded. The push rod 34 is then free to be slid relative to the hollow needle 32 to eject the therapeutic elements. The simple economical design of the stopper 10 permits it to be discarded without adding any significant cost to the procedure.

Although the invention has been disclosed in its preferred forms, the specific embodiments thereof as disclosed and illustrated herein are not to be considered in a limiting sense, because numerous variations are possible. The subject matter of the invention includes all novel and non-obvious combinations and subcombinations of the various elements, features, functions, and/or properties disclosed herein.

What is claimed is:

1. A stopper for resisting movement of a slideable stylet within a cannula, comprising:
a planar body having first and second major surfaces and a protruding portion,
the protruding portion having a first surface for frictionally engaging the stylet relative to the cannula.

2. The stopper of claim 1, wherein protruding portion includes a tapered portion.

3. The stopper of claim 2, wherein the cannula includes a first end having a tapered inner surface.

4. The stopper of claim 3, wherein the tapered portion of the stopper is frictionally engageable with the tapered inner surface of the first end of the cannula.

5. The stopper of claim 1, wherein the protruding portion further comprises a groove.

6. The stopper of claim 5, wherein the groove is adapted for receiving the stylet.

7. The stopper of claim 5, wherein the groove is sized to releaseably grip the stylet.

8. A surgical needle assembly that comprises:
a hollow needle with an opening on a distal end of the needle and a gripping member on the proximal end of the needle, the gripping member including an inner surface;
a push rod inserted into the hollow needle; and
a stopper having a protruding portion removeably disposed between the push rod and the inner surface of the gripping member.

9. The surgical needle assembly of claim 8, wherein the stopper can be removed from the assembly without disturbing the position of the push rod.

10. The surgical needle assembly of claim 8, wherein the stopper comprises a planar body connected to the protruding portion.

11. The surgical needle assembly of claim 8, wherein the protruding portion has a tapered shape.

12. The surgical needle assembly of claim 11, wherein the inner surface of the gripping member includes a tapered inner surface.

13. The surgical needle assembly of claim 12, wherein the tapered shape of the protruding portion is sized such that the protruding portion and the push rod fit snugly within the tapered inner surface of the gripping member.

14. The surgical needle assembly of claim 10, wherein the stopper has a groove that extends from a base of the protruding portion to a top edge of the planar body.

15. The surgical needle assembly of claim 10, wherein the planar body is configured for gripping by a finger or thumb.

16. The surgical needle assembly of claim 14, wherein the groove is adapted for receiving the push rod.

17. The surgical needle assembly of claim 14, wherein the groove is sized to releaseably grip the push rod.

18. The surgical needle assembly of claim 14, wherein the groove fits almost concentrically around the push rod and the gripping member fits concentrically around the protruding portion.

19. The stopper of claim 5, wherein the groove fits almost concentrically around the stylet and a first end of the cannula fits concentrically around the protruding portion.
